(19) United States
(12) Patent Application Publication
(10) Pub. No.: US 2026/0083468 A1
(43) Pub. Date: Mar. 26, 2026

(54) FOUR-INTERFACE SHOCKWAVE BALLOON CATHETER AND ITS CONTROL SYSTEM

(71) Applicant: VASCUPATENT MEDICAL (SHENZHEN) CO., LTD., Shenzhen (CN)

(72) Inventors: Junxiong OUYANG, Shenzhen (CN); Lizhong LU, Shenzhen (CN); Xueqi LI, Shenzhen (CN)

(21) Appl. No.: 18/893,982

(22) Filed: Sep. 24, 2024

(51) Int. Cl. A61B 17/22; A61M 25/10

(52) U.S. Cl. CPC A61B 17/22012; A61M 25/10; A61M 25/10181; A61M 25/10185; A61M 25/10186; A61B 2017/22025; A61B 2017/22062; A61M 2025/1079; A61M 2025/109; A61M 2205/75

(57) ABSTRACT

The present invention provides a four-interface shock wave balloon catheter and its control system, based on the design of the four-interface catheter base structure and the corresponding catheter structure, have separate interfaces specifically designed for replenishing new fluid and discharge of fluid, enabling simultaneous replenishment and cyclic replacement of fluid within the balloon. This allows the balloon to remain in an expanded state and ensures the stability of the acoustic impedance of the fluid within the balloon as solid precipitates are removed along with the fluid, and helps to ensure the effectiveness of the surgical treatment and shorten the surgical time, and by shortening the surgical time can reduce the occurrence of intraoperative accidents and avoid surgical risks arising from the repeated inflation and deflation of the balloon 23, thereby improving the safety of the surgery.

### BACKGROUND OF THE PRESENT INVENTION

### FIELD OF INVENTION

The present invention relates to a medical device, and particularly relates to a four-interface shock wave balloon catheter and its control system.

### DESCRIPTION OF RELATED ARTS

Cardiovascular diseases have long been one of the major threats to human health and life, and with the changes in modern lifestyles, the prevalence is increasing and affecting younger populations. Among these, diffuse vascular calcification is a major cause of severe cardiovascular diseases such as coronary heart disease and stroke. This condition is characterized by the proliferation of fibrous tissue in the vascular intima and the deposition of calcium, leading to thickened and hardened blood vessels and narrowed vascular lumen. Once the lumen becomes completely blocked, ischemia or even necrosis in the tissues or organs supplied by the blood vessels will occur. For severe diffuse calcification of the vascular wall, a surgical treatment method using a cutting balloon is generally adopted. However, this surgical treatment method can cause damage to the endothelial cells of the inner layer of the vessel during balloon expansion.

To address this, shock wave balloon catheter is developed to treat diffuse calcification in the vessel wall. After the balloon of the shock wave balloon catheter is delivered to the calcified site in the vessel wall, the balloon is inflated and expanded. Then, a high-voltage pulse power source is activated to release a high-voltage pulse to the shock wave generation device of the shock wave balloon catheter to generate intermittent shock waves, thereby shattering the calcified plaque at the calcified site in the vessel wall and achieving the purpose of smoothening blood flow. Specifically, the existing shock wave balloon catheter adopts a three-interface design, of which one interface is used for inflation and deflation of the balloon, and the other two interfaces are respectively used for introducing a guide wire and connecting to a high-voltage pulse. In order to achieve the therapeutic effect, the shock wave balloon catheter needs to be maintained in a state of inflation that is tightly pressed against the vessel wall for a relatively long time (several minutes or even tens of minutes), so as to continuously emit shock waves and transmit them to the calcified site in the vessel wall.

However, on the one hand, the balloon catheter continuously generates gas bubbles during the emission of shock waves, which cannot be discharged in time and accumulate and dissolve in the catheter fluid, leading to changes in the acoustic impedance of the fluid. This hinders the effective transmission of shock waves and prevents the achievement of the desired therapeutic effect. On the other hand, the shock wave emitted within the catheter is generated by electric arc discharge. Electric arc discharge will produce high temperatures and release plasma. The plasma is cooled into solid particles in the catheter fluid, while the electrodes will undergo thermal erosion and release metal ions. When these metal ions enter the fluid, they undergo oxidation-reduction reactions and also form solid precipitates. These solid substances cannot be discharged in time and further affect the acoustic impedance of the fluid, thus hindering the transmission of the shock wave and affecting the therapeutic effect. Existing techniques for solving the problem of solid precipitation involve triggering a fixed number of shock waves, then deflating and draining the balloon, and finally refilling the balloon with new fluid to remove the solid precipitates.

Additionally, the prolonged contact of the balloon catheter with the vessel wall may lead to blood flow blockage and distal vessel ischemia. Therefore, the operator usually needs to set intermittent pause times during the procedure and use these pause times to deflate and drain the balloon catheter to remove solid precipitates before re-inflating and expanding it for shock wave therapy. However, due to multiple inflations and deflations, the balloon catheter is prone to displacement, which can lead to inaccurate targeting of the treatment site and reduce the therapeutic effect, and setting intermittent pause times can also extend the overall treatment time, increasing the risk of intraoperative infections and other complications. Furthermore, multiple inflations and deflations can also affect the elasticity of the catheter, potentially leading to catheter rupture, resulting in a serious intraoperative complication. Additionally, the repeated contraction and expansion of the catheter can damage the vessel wall, leading to thrombosis or even death.

### SUMMARY OF THE PRESENT INVENTION

The objective of the present invention is to provide a four-interface shock wave balloon catheter and its control system, the four-interface shock wave balloon catheter and its control system can maintain the acoustic impedance of the fluid within the balloon in a stable state during balloon expansion. This helps to ensuring the effectiveness of the surgical treatment and shorten the surgical time.

Another purpose of the present invention is to provide a four-interface shock wave balloon catheter and its control system, the four-interface shock wave balloon catheter and its control system can shorten the surgical time while ensuring the effectiveness of the surgical treatment, thus contributing to improving surgical efficiency, reducing surgical costs, and benefiting from the shortened surgical time to reduce the occurrence of intraoperative accidents and improve surgical safety.

Another purpose of the present invention is to provide a four-interface shock wave balloon catheter and its control system, the four-interface shock wave balloon catheter and its control system can maintain the acoustic impedance of the fluid within the balloon in a stable state during balloon expansion, thus contributing to avoiding the surgical risks associated with repeated inflation and deflation of the balloon, and thereby ensuring surgical safety.

Another purpose of the present invention is to provide a four-interface shock wave balloon catheter and its control system, the four-interface shock wave balloon catheter and its control system can simultaneously drain the fluid within the balloon while replenishing it with new fluid, thus, achieving the cyclic replacement of the fluid within the balloon. This allows the balloon to remain in an expanded state and ensures the stability of the acoustic impedance of the fluid within the balloon as solid precipitates are removed along with the fluid.

Another purpose of the present invention is to provide a four-interface shock wave balloon catheter and its control system, the four-interface shock wave balloon catheter and its control system, based on the design of the four-interface catheter base structure and the corresponding catheter structure, have separate interfaces specifically designed for replenishing new fluid and draining fluid. This enables simultaneous replenishment and discharge of fluid, and based on these separate interfaces for replenishment and drainage, it realizes the cyclic replacement of fluid within the balloon, thereby maintaining a fresh liquid environment within the balloon.

According to one aspect of the present invention, the present invention provides a four-interface shock wave balloon catheter, the four-interface shock wave balloon catheter comprises:

A catheter base, the catheter base is designed with a four-interface structure and has a fluid replenishment interface, a fluid discharge interface, a guide wire interface, a pulse interface, and a catheter connection end; and

A shock wave balloon catheter, the shock wave balloon catheter comprises a guide wire inner tube, an outer tube, a balloon, a fluid discharge tube, and at least two shock wave emitters, wherein when the shock wave balloon catheter is connected to the catheter connection end of the catheter base, the outer tube is connected to the catheter connection end, the guide wire inner tube communicates with the guide wire interface through the catheter connection end, and extends out of the outer tube through the catheter connection end, forming a fluid replenishment channel between the guide wire inner tube and the outer tube, which communicates with the fluid replenishment interface through the catheter connection end, wherein the part of the guide wire inner tube that extends out of the outer tube is the distal segment of the guide wire inner tube, one end of the balloon is sealedly connected to the end of the outer tube that is farther from the catheter connection end, and the other end of the balloon is sealedly connected to the distal segment of the guide wire inner tube, forming a balloon chamber that communicates with the fluid replenishment channel, wherein the fluid discharge tube communicates with the fluid discharge interface through the catheter connection end, and into the balloon chamber along the guide wire inner tube, wherein the at least two shock wave emitters are arranged on the distal segment of the guide wire inner tube in the balloon chamber, the conducting wires of the at least two shock wave emitters, while being insulating isolated from the balloon chamber and the fluid replenishment channel, are arranged along the guide wire inner tube, and are led out through the catheter connection end at the pulse interface.

In one embodiment, wherein the fluid discharge tube, when arranged within the guide wire inner tube, extends from the catheter connection end along the guide wire inner tube into the balloon chamber.

In one embodiment, wherein the end of the fluid discharge tube that extends into the balloon chamber is positioned close to the end where the balloon is sealedly connected to the distal segment.

In one embodiment, wherein each of the shock wave emitters comprises a metal ring and an insulating layer, wherein the shock wave emitter is arranged within the guide wire inner tube with the metal ring annularly disposed around the distal segment, and the insulating layer, while the metal ring is annularly disposed around the distal segment, provides isolation between the metal ring and the distal segment, the metal ring is arranged with two emission holes that penetrate through its ring wall on opposite sides of its ring wall, the insulating layer is hollowed-out arranged at the positions corresponding to the two emission holes, wherein the number of the conducting wires of each of the shock wave emitters is two, the two conducting wires, while being insulating isolated from the metal ring by the insulating layer, extend from the two emission holes of the metal ring along the guide wire inner tube and are led out through the catheter connection end at the pulse interface, respectively.

In one embodiment, wherein the alignment direction of the two emission holes of each of the shock wave emitters determines the orientation of the emission holes. The adjacent shock wave emitters are arranged around the guide wire inner tube with a rotational offset along the axial direction, resulting in different orientations of the emission holes.

In one embodiment, wherein the number of the shock wave emitters is two, and the emission holes of the two shock wave emitters are arranged with a rotational offset of 90° along the axial direction of the guide wire inner tube, resulting in the emission holes that are oriented at a 90° angle to each other.

In one embodiment, wherein the fluid discharge tube is arranged along the guide wire inner tube with an offset relative to the emission holes of the shock wave emitter.

In one embodiment, wherein the portion of the fluid discharge tube that extends into the balloon chamber is staggered relative to the emission holes when arranged around the guide wire inner tube.

In one embodiment, wherein the location where the shock wave emitters are arranged in the guide wire inner tube, is arranged with an insulating isolation layer that provides isolation between the conducting wires of the shock wave emitters and the distal segment.

In one embodiment, wherein the insulating isolation layer is arranged in the guide wire inner tube with the insulating isolation layer covering the guide wire inner tube.

In one embodiment, wherein the guide wire inner tube further comprises an insulating medium that covers the conducting wires, based on the insulating layer, the insulating isolation layer and the insulating medium, the portions of the conducting wires of the shock wave emitters, except for the portion that exposed to the emission holes, are insulating isolated from the balloon chamber and the fluid replenishment channel.

In one embodiment, wherein the insulating medium is arranged in the guide wire inner tube with the insulating medium covering the guide wire inner tube.

In one embodiment, wherein the fluid discharge tube is arranged within the guide wire inner tube with the insulating medium covering the guide wire inner tube in a segmented manner.

In one embodiment, wherein the shock wave balloon catheter further comprises at least one contrast marker which is directly or indirectly fixed to the distal segment within the balloon chamber, to monitor the position of the balloon during the procedure and ensure the balloon is located in the target calcified area of the blood vessel, based on the visualization of the contrast marker.

In one embodiment, wherein the number of the contrast markers is two, the two contrast markers are directly or indirectly fixed to the distal segment at positions near the two opposite ends of the balloon within the balloon chamber, respectively.

In one embodiment, wherein the two contrast markers are arranged in a ring-shaped manner and are annularly disposed around the distal segment of the guide wire inner tube.

In one embodiment, wherein the contrast marker located near the end where the balloon is sealedly connected to the distal segment is fixed to the guide wire inner tube, while the other contrast marker, located near the end where the balloon is sealedly connected to the outer tube, is annularly disposed around the guide wire inner tube while being adhesively fixed to the insulating medium.

In one embodiment, wherein the guide wire interface of the catheter base is arranged coaxially with the catheter connection end, while the fluid replenishment interface, the fluid discharge interface, and the pulse interface are arranged laterally relative to the guide wire interface and the catheter connection end that are arranged coaxially, and are arranged such that the pulse interface is closer to the guide wire interface relative to the fluid replenishment interface and the fluid discharge interface.

According to another aspect of the present invention, the present invention provides a control system of a four-interface shock wave balloon catheter comprises:

A catheter base, the catheter base is designed with a four-interface structure and has a fluid replenishment interface, a fluid discharge interface, a guide wire interface, a pulse interface, and a catheter connection end;

A shock wave balloon catheter, the shock wave balloon catheter comprises a guide wire inner tube, an outer tube, a balloon, a fluid discharge tube, and at least two shock wave emitters, wherein when the shock wave balloon catheter is connected to the catheter connection end of the catheter base, the outer tube is connected to the catheter connection end, the guide wire inner tube communicates with the guide wire interface through the catheter connection end, and extends out of the outer tube through the catheter connection end, forming a fluid replenishment channel between the guide wire inner tube and the outer tube, which communicates with the fluid replenishment interface through the catheter connection end, wherein the part of the guide wire inner tube that extends out of the outer tube is the distal segment of the guide wire inner tube, one end of the balloon is sealedly connected to the end of the outer tube that is farther from the catheter connection end, and the other end of the balloon is sealedly connected to the distal segment of the guide wire inner tube, forming a balloon chamber that communicates with the fluid replenishment channel., wherein the fluid discharge tube communicates with the fluid discharge interface through the catheter connection end, and extends into the balloon chamber along the guide wire inner tube, wherein the at least two shock wave emitters are arranged on the distal segment of the guide wire inner tube in the balloon chamber, the conducting wires of the at least two shock wave emitters, while being insulating isolated from the balloon chamber and the fluid replenishment channel, are arranged along the guide wire inner tube, and are led out through the catheter connection end at the pulse interface;and

A circulation control device, wherein the circulation control device comprises a fluid replenishment mechanism and a recovery mechanism, the fluid replenishment mechanism comprises a constant pressure peristaltic pump and a pressurizing pump, wherein the constant pressure peristaltic pump has a fluid replenishment port and an output port, and the output ports of the pressurizing pump and the constant pressure peristaltic pump are connected to the fluid replenishment interface of the catheter base through a three-way connector, wherein the pipeline of the pressurizing pump that connects the three-way connector is arranged with a three-way valve configured as an electromagnetic valve, wherein two of the interfaces of the three-way valve are respectively connected to the pressurizing pump and the three-way connector, and the third interface of the three-way valve serves as an air inlet, on the connecting pipe between the three-way connector and the output port of the constant pressure peristaltic pump, a first electromagnetic valve is installed to control the opening and closing of this connecting pipeline, wherein a pressure sensor is installed on the connecting pipeline between the three-way connector with the first electromagnetic valve and the output port of the constant pressure peristaltic pump, the recovery mechanism comprises a one-way valve and a second electromagnetic valve, wherein the inlet of the one-way valve is connected to the fluid discharge interface of the catheter base, the outlet of the one-way valve is connected to one of the interfaces of the second electromagnetic valve, and the other interface of the second electromagnetic valve serves as a fluid discharge port.

In one embodiment, the fluid discharge port is directly or indirectly connected to the fluid replenishment port of the constant pressure peristaltic pump through a filtering device, to achieve recycling and reuse by filtering the waste fluid.

Through the understanding of the following description and drawings, the further objectives and advantages of the present invention will be fully demonstrated.

### BRIEF DESCRIPTION OF THE DRAWINGS

FIG. 1 is a schematic structural view of a four-interface shock wave balloon catheter control system according to one embodiment of the present invention.

FIG. 2 is a partial axial sectional schematic view of the four-interface shock wave balloon catheter of the above embodiment of the present invention.

FIG. 3 is a partial axial sectional schematic view of the four-interface shock wave balloon catheter of the above embodiment of the present invention.

FIG. 4 is a radial sectional schematic view of the segment of the shock wave balloon catheter of the four-interface shock wave balloon catheter of the above embodiment of the present invention with the outer tube.

FIG. 5 is a partial three-dimensional sectional schematic view of the four-interface shock wave balloon catheter of the above embodiment of the present invention.

FIG. 6 is a partial three-dimensional sectional schematic view of the four-interface shock wave balloon catheter of the above embodiment of the present invention.

### DETAILED DESCRIPTION OF THE PREFERRED EMBODIMENT

The following description is used to disclose the present invention so that those skilled in the art can implement the present invention. The preferred embodiments in the following description are only examples, and those skilled in the art can think of other obvious variations. The basic principles of the present invention defined in the following description can be applied to other embodiments, modifications, improvements, equivalents, and other technical solutions without departing from the spirit and scope of the present invention.

It should be understood that the term “a” should be interpreted to mean “at least one” or “one or more.” In one embodiment, the number of an element can be one, while in another embodiment, the number of that element can be multiple. The term “a” should not be interpreted as a limitation on quantity.

The present invention provides a four-interface shock wave balloon catheter and its control system. The four-interface shock wave balloon catheter and control system can maintain the acoustic impedance of the fluid within the balloon in a stable state during balloon expansion. This helps to ensure the effectiveness of the surgical treatment and shorten the surgical time.

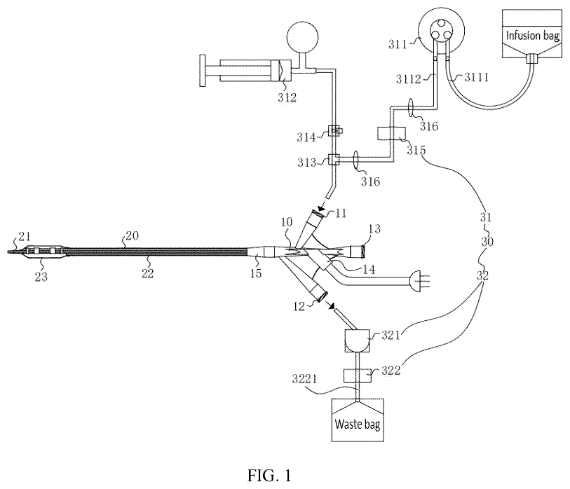

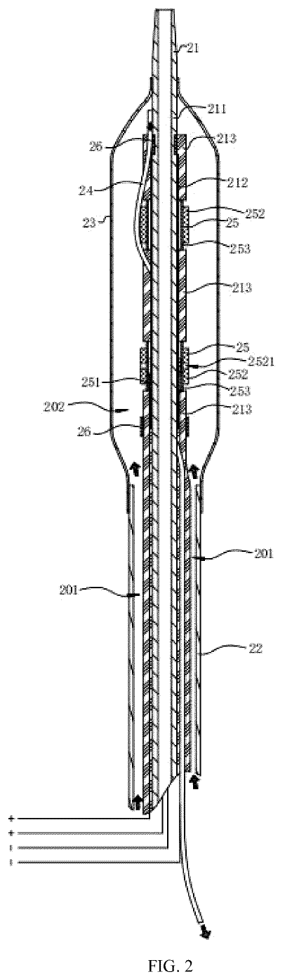

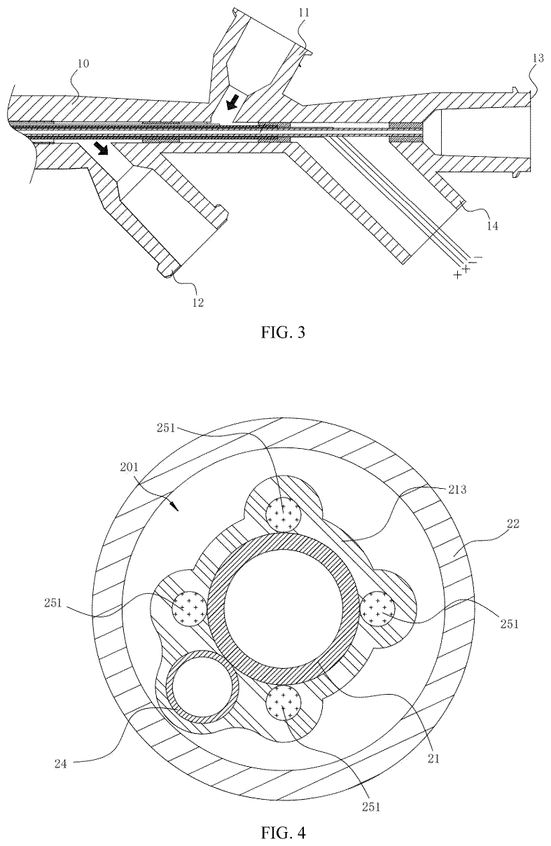

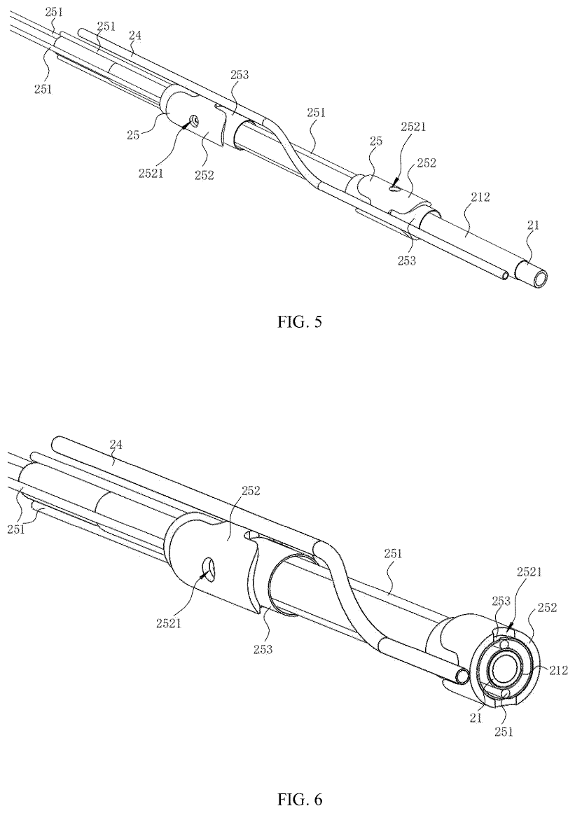

Specifically, referring to FIGS. 1 to 6 of the description drawing of present invention, according one embodiment of the structure of the four-interface shock wave balloon catheter is illustrated. The four-interface shock wave balloon catheter comprises a catheter base 10 and a shock wave balloon catheter 20, wherein the catheter base 10 is designed with a four-interface structure and has a fluid replenishment interface 11, a fluid discharge interface 12, a guide wire interface 13, a pulse interface 14, and a catheter connection end 15. The shock wave balloon catheter 20 comprises a guide wire inner tube 21, an outer tube 22, a balloon 23, a fluid discharge tube 24, and at least two shock wave emitters 25, wherein when the shock wave balloon catheter 20 is connected to the catheter connection end 15 of the catheter base 10, the outer tube 22 is connected to the catheter connection end 15. The guide wire inner tube 21 communicates with the guide wire interface 13 through the catheter connection end 15, and extends out of the outer tube 22 through the catheter connection end 15, forming a fluid replenishment channel 201 between the guide wire inner tube 21 and the outer tube 22, which communicates with the fluid replenishment interface 11 through the catheter connection end 15, wherein the part of the guide wire inner tube 21 that extends out of the outer tube 22 is the distal segment 211 of the guide wire inner tube 21. One end of the balloon 23 is sealedly connected to the end of the outer tube 22 that is farther from the catheter connection end 15, and the other end of the balloon 23 is sealedly connected to the distal segment 211 of the guide wire inner tube 21, forming a balloon chamber 202 that communicates with the fluid replenishment channel 201, wherein the fluid discharge tube 24 communicates with the fluid discharge interface 12 through the catheter connection end 15, and extends into the balloon chamber 202 along the guide wire inner tube 21. Wherein the at least two shock wave emitters 25 are arranged on the distal segment 211 of the guide wire inner tube 21 in the balloon chamber 202. The conducting wires 251 of the at least two shock wave emitters 25, while being insulating isolated from the balloon chamber 202 and the fluid replenishment channel 201, are arranged along the guide wire inner tube 21, and are led out through the catheter connection end 15 at the pulse interface 14. Therefore, the fluid replenishment interface 11 can simultaneously replenish new fluid while the fluid discharge interface 12 discharges fluid, thereby achieving the circulation and replacement of the liquid within the balloon chamber 202. This corresponds to maintain the balloon 23 in an expanded state, and maintaining the stability of the acoustic impedance of the fluid within the balloon chamber 202 while the balloon 23 is in the expanded state and the solid precipitates within the balloon chamber 202 are discharged. Thus, when the at least two shock wave emitters 25 are connected to an external pulse power source through the guide wire 251 and the pulse interface 14, the efficiency of the shock waves emitted by the at least two shock wave emitters 25 in the fluid within the balloon chamber 202 can be ensured, thereby ensuring the stability of the intraoperative treatment effect.

It is worth noting that the four-interface shock wave balloon catheter can maintain the acoustic impedance of the fluid within the balloon chamber 202 in a stable state during the expansion of the balloon 23, thus ensuring the stability of the intraoperative treatment effect. This is beneficial for shortening the surgical time while ensuring the effectiveness of the surgical treatment, which in turn helps to improve surgical efficiency and reduce surgical costs, and by shortening the surgical time can reduce the occurrence of intraoperative accidents and avoid surgical risks arising from the repeated inflation and deflation of the balloon 23, thereby improving the safety of the surgery.

For example, in this embodiment of the present invention, the balloon 23 is in a cylindrical shape when expanded. Correspondingly, the balloon 23 is made of an expandable material, such as a nylon elastic contraction material. The guide wire inner tube 21 is made of an insulating material, such as HDPE, and the outer tube 22 is made of a nylon material. Correspondingly, the guide wire inner tube 21 and the balloon 23 can be sealedly connected through laser welding, and the outer tube 22 and the balloon 23 can be sealedly connected through heat welding.

Furthermore, in this embodiment of the present invention, the fluid discharge tube 24, when arranged within the guide wire inner tube 21, extends from the catheter connection end 15 along the guide wire inner tube 21 into the balloon chamber 202. This can reduce the disturbance of the fluid within the balloon chamber 202 by the fluid discharge tube 24 in the fluid discharge state, thus helping to ensure the stability of the acoustic impedance of the fluid within the balloon chamber 202 and the stability of the intraoperative treatment effect.

Specially, in this embodiment of the present invention, the end of the fluid discharge tube 24 that extends into the balloon chamber 202 is positioned close to the end where the balloon 23 is sealedly connected to the distal segment 211. This ensures that the positions for replenishing new fluid and discharging fluid within the balloon chamber 202 respectively correspond to the two opposite ends of the balloon 23, thereby facilitating the circulation of the fluid within the balloon chamber 202 and maintains the acoustic impedance of the fluid within the balloon chamber 202 at different positions. This correspond to help ensuring the transmission efficiency of the shock waves emitted by the shock wave emitters 25 located at different positions within the balloon chamber 202, thereby ensuring the stability of the intraoperative treatment effect.

It is worth noting that, in this embodiment of the present invention, the portion of the fluid discharge tube 24 that extends into the balloon chamber 202 is arranged around the guide wire inner tube 21. This further reduces the disturbance of the fluid within the balloon chamber 202 by the fluid discharge tube 24 in the fluid discharge state, thereby helping to further ensure the stability of the acoustic impedance of the fluid within the balloon chamber 202 and the stability of the intraoperative treatment effect.

Furthermore, in this embodiment of the present invention, each of the shock wave emitters 25 comprises a metal ring 252 and an insulating layer 253, wherein the shock wave emitter 25 is arranged within the guide wire inner tube 21 with the metal ring 252 annularly disposed around the distal segment 211, and the insulating layer 253, while the metal ring 252 is annularly disposed around the distal segment 211, provides isolation between the metal ring 252 and the distal segment 211, wherein the metal ring 252 is arranged with two emission holes 2521 that penetrate through its ring wall on opposite sides of its ring wall, wherein the insulating layer 253 is hollowed-out arranged at the positions corresponding to the two emission holes 2521, wherein the number of the conducting wires 251 of each of the shock wave emitters 25 is two, the two conducting wires 251, while being insulating isolated from the metal ring 252 by the insulating layer 253, extend from the two emission holes 2521 of the metal ring 252 along the guide wire inner tube 21 and are led out through the catheter connection end 15 at the pulse interface 14, respectively. This exposes one end of each of the guide wires 251 to the corresponding emission hole 2521 of the metal ring 252, allowing the shock waves emitted by each of the shock wave emitters 25 to be released in a fixed direction from the two emission holes 2521 of the metal ring 252, thereby avoiding the problem of excessive shock wave range during surgery and improving the safety of the surgical procedure.

Specially, in this embodiment of the present invention, the alignment direction (i.e., the penetration direction) of the two emission holes 2521 of each of the shock wave emitters 25 determines the orientation of the emission holes 2521. The adjacent shock wave emitters 25 are arranged around the guide wire inner tube 21 with a rotational offset along the axial direction, resulting in different orientations of the emission holes. This helps to balance the range and energy distribution of the shock waves emitted by the shock wave emitters 25 during the surgical procedure, thereby helping to ensure the effectiveness of the intraoperative treatment and improve the safety of the surgery.

Specifically, in this embodiment of the present invention, the number of the shock wave emitters 25 is two, and the emission holes 2521 of the two shock wave emitters 25 are arranged with a rotational offset of 90° along the axial direction of the guide wire inner tube 21, resulting in the emission holes 2521 that are oriented at a 90° angle to each other.

It is worth noting that, in this embodiment of the present invention, the fluid discharge tube 24 is arranged along the guide wire inner tube 21 with an offset relative to the emission holes 2521 of the shock wave emitter 25. This helps to avoid the fluid discharge tube 24 obstructing the emission of the shock wave emitter 25.

Specifically, in this embodiment of the present invention, the portion of the fluid discharge tube 24 that extends into the balloon chamber 202 is staggered relative to the emission holes 2521 when arranged around the guide wire inner tube 21. This helps to avoid the fluid discharge tube 24 obstructing the emission of the shock wave emitter 25 while reducing the disturbance of the fluid within the balloon chamber 202 by the fluid discharge tube 24 in the fluid discharge state. This helps to ensure the stability of the intraoperative treatment effect.

Specially, in this embodiment of the present invention, the location where the shock wave emitters 25 are arranged in the guide wire inner tube 21, is arranged with an insulating isolation layer 212 that provides isolation between the conducting wires 251 of the shock wave emitters 25 and the distal segment 211. Based on this arrangementof the insulating isolated layer 212, the mobility of the guide wire inner tube 21 is ensured while preventing the guide wire 251 of the shock wave emitter 25 from damaging the guide wire inner tube 21 during the discharge behavior at the emission hole 2521.

Specifically, in this embodiment of the present invention, the insulating isolation layer 212 is arranged in the guide wire inner tube 21 with the insulating isolation layer 212 covering the guide wire inner tube 21.

For example, in this embodiment of the present invention, the insulating layer 253 and the insulating isolation layer 212 are both made of PI (polyimide) material.

Furthermore, the guide wire inner tube 21 further comprises an insulating medium 213 that covers the conducting wires 251, based on the insulating layer 253, the insulating isolation layer 212 and the insulating medium 213, the portions of the conducting wires 251 of the shock wave emitters 25, except for the portion that exposed to the emission holes 2521, are insulating isolated from the balloon chamber 202 and the fluid replenishment channel 201.

Specifically, in this embodiment of the present invention, the insulating medium 213 is arranged in the guide wire inner tube 21 with the insulating medium 213 covering the guide wire inner tube 21.

Therefore, the insulating medium 213 and the insulating isolation layer 212 can either be separate layers formed through different processes or a single, inseparable structure formed through the same or different processes. However, this does not affect the definition of the insulating isolation layer 212 and the insulating medium 213 described above.

Furthermore, in this embodiment of the present invention, the fluid discharge tube 24 is arranged within the guide wire inner tube 21 with the insulating medium 213 covering the guide wire inner tube 21 in a segmented manner.

Specifically, in this embodiment of the present invention, the insulating medium 213 is a heat shrinking tube made of PE material, which is arranged with a plurality of channels that can independently pass through a single number of the fluid discharge tube 24, the guide wire inner tube 21, and each of the guide wires 251.

Furthermore, in this embodiment of the present invention, the guide wire interface 13 of the catheter base 10 is arranged coaxially with the catheter connection end 15, while the fluid replenishment interface 11, the fluid discharge interface 12, and the pulse interface 14 are arranged laterally relative to the guide wire interface 13 and the catheter connection end 15 that are arranged coaxially, and are specifically arranged such that the pulse interface 14 is closer to the guide wire interface 13 relative to the fluid replenishment interface 11 and the fluid discharge interface 12. This arrangement ensures the smooth insertion of the guide wire through the guide wire port 13 into the guide wire inner tube 21 and the safe connection of the external pulse power source to the pulse port 14.

Furthermore, in this embodiment of the present invention, the shock wave balloon catheter 20 further comprises at least one contrast marker 26 which is directly or indirectly fixed to the distal segment 211 within the balloon chamber 202, to monitor the position of the balloon 23 during the procedure and ensure the balloon 23 is located in the target calcified area of the blood vessel, based on the visualization of the contrast marker 26.

Specifically, in this embodiment of the present invention, the number of the contrast markers 26 is two, the two contrast markers 26 are directly or indirectly fixed to the distal segment 211 at positions near the two opposite ends of the balloon 23 within the balloon chamber 202, respectively. This allows for precise monitoring of the position of the balloon 23 during intraoperative visualization, as the position of the balloon 23 is corresponded to the region between the two contrast markers 26.

Furthermore, in this embodiment of the present invention, the two contrast markers 26 are arranged in a ring-shaped manner and are annularly disposed around the distal segment 211 of the guide wire inner tube 21. This helps to ensure the visibility of the contrast markers 26 from various angles.

Specifically, in this embodiment of the present invention, the contrast marker 26 located near the end where the balloon 23 is sealedly connected to the distal segment 211 is fixed to the guide wire inner tube 21 by swaging, while the other contrast marker 26, located near the end where the balloon 23 is sealedly connected to the outer tube 22, is annularly disposed around the guide wire inner tube 21 while being adhesively fixed to the insulating medium 213.

To further understand the present invention, according to the above embodiment of the present invention, the four-interface shock wave balloon catheter control system is illustrated in FIG. 1, wherein when using the four-interface shock wave balloon catheter, the control system of the four-interface shock wave balloon catheter further comprises a circulation control device 30, wherein the circulation control device comprises a fluid replenishment mechanism 31 and a recovery mechanism 32, wherein the fluid replenishment mechanism 31 comprises a constant pressure peristaltic pump 311 and a pressurizing pump 312, wherein the constant pressure peristaltic pump 311 has a fluid replenishment port 3111 and an output port 3112, and the output ports 3112 of the pressurizing pump 312 and the constant pressure peristaltic pump 311 are connected to the fluid replenishment interface 11 of the catheter base 10 through a three-way connector 313, wherein the pipeline of the pressurizing pump 312 that connects the three-way connector 313 is arranged with a three-way valve 314 configured as an electromagnetic valve, wherein two of the interfaces of the corresponded three-way valve 314 are respectively connected to the pressurizing pump 312 and the three-way connector 313, and the third interface of the three-way valve 314 serves as an air inlet. On the connecting pipe between the three-way connector 313 and the output port 3112 of the constant pressure peristaltic pump 311, a first electromagnetic valve 315 is installed to control the opening and closing of this connecting pipeline, wherein a pressure sensor 316 is installed on the connecting pipelines between the three-way connector 313 with the first electromagnetic valve 315 and the output port 3112 of the constant pressure peristaltic pump 311, respectively; the recovery mechanism 32 comprises a one-way valve 321 and a second electromagnetic valve 322, wherein the inlet of the one-way valve 321 is connected to the fluid discharge interface 12 of the catheter base 10, the outlet of the one-way valve 321 is connected to one of the interfaces of the second electromagnetic valve 322, and the other interface of the second electromagnetic valve 322 serves as a fluid discharge port 3221.

It is worth noting that, in this embodiment of the present invention, the fluid discharge port 3221 can optionally be directly or indirectly connected to the fluid replenishment port 3111 of the constant pressure peristaltic pump 311 through a filtering device, to achieve recycling and reuse by filtering the waste fluid. For example, as shown in FIG. 1, when an an infusion bag is connected to the fluid replenishment port 3111 of the constant pressure peristaltic pump 311, the fluid discharge port 3221 communicates with the infusion bag through a filtering device, allowing it to communicate with the fluid replenishment port of the pressure-stabilized peristaltic pump.

Those skilled in the art will understand that the embodiments and examples described above, as well as the drawings illustrating the principles of the invention, are merely illustrative and do not limit the invention. The objectives of the invention have been fully and effectively realized. The functions and structural principles of the invention have been shown and described in the embodiment, and without departing from these principles, the invention can be embodied in various forms and modification.

What is claimed is:

1. A four-interface shock wave balloon catheter, comprising:
a catheter base, said catheter base is designed with a four-interface structure and has a fluid replenishment interface, a fluid discharge interface, a guide wire interface, a pulse interface, and a catheter connection end; and
a shock wave balloon catheter, said shock wave balloon catheter comprises a guide wire inner tube, an outer tube, a balloon, a fluid discharge tube, and at least two shock wave emitters, wherein when said shock wave balloon catheter is connected to said catheter connection end of said catheter base, said outer tube is connected to said catheter connection end, said guide wire inner tube communicates with said guide wire interface through said catheter connection end, and extends out of said outer tube through said catheter connection end, forming a fluid replenishment channel between said guide wire inner tube and said outer tube, which communicates with said fluid replenishment interface through said catheter connection end, wherein the part of said guide wire inner tube that extends out of said outer tube is the distal segment of said guide wire inner tube, one end of said balloon is sealedly connected to the end of said outer tube that is farther from said catheter connection end, and the other end of said balloon is sealedly connected to said distal segment of said guide wire inner tube, forming a balloon chamber that communicates with said fluid replenishment channel, wherein said fluid discharge tube communicates with said fluid discharge interface through said catheter connection end, and into said balloon chamber along said guide wire inner tube, wherein said at least two shock wave emitters are arranged on said distal segment of said guide wire inner tube in said balloon chamber, said conducting wires of said at least two shock wave emitters, while being insulating isolated from said balloon chamber and said fluid replenishment channel, are arranged along said guide wire inner tube, and are led out through said catheter connection end at said pulse interface.

2. The four-interface shock wave balloon catheter, as recited in claim 1, wherein said fluid discharge tube, when arranged within said guide wire inner tube, extends from said catheter connection end along said guide wire inner tube into said balloon chamber.

3. The four-interface shock wave balloon catheter, as recited in claim 2, wherein the end of said fluid discharge tube that extends into said balloon chamber is positioned close to the end where said balloon is sealedly connected to said distal segment.

4. The four-interface shock wave balloon catheter, as recited in claim 3, wherein each of said shock wave emitters comprises a metal ring and an insulating layer, wherein said shock wave emitter is arranged within said guide wire inner tube with said metal ring annularly disposed around said distal segment, and said insulating layer, while said metal ring is annularly disposed around said distal segment, provides isolation between said metal ring and said distal segment, said metal ring is arranged with two emission holes that penetrate through its ring wall on opposite sides of its ring wall, said insulating layer is hollowed-out arranged at the positions corresponding to said two emission holes, wherein the number of said conducting wires of each of said shock wave emitters is two, said two conducting wires, while being insulating isolated from said metal ring by said insulating layer, extend from said two emission holes of said metal ring along said guide wire inner tube and are led out through said catheter connection end at said pulse interface, respectively.

5. The four-interface shock wave balloon catheter, as recited in claim 4, wherein the alignment direction of said two emission holes of each of said shock wave emitters determines the orientation of said emission holes, said adjacent shock wave emitters are arranged around said guide wire inner tube with a rotational offset along the axial direction, resulting in different orientations of said emission holes.

6. The four-interface shock wave balloon catheter, as recited in claim 5, wherein the number of said shock wave emitters is two, and said emission holes of said two shock wave emitters are arranged with a rotational offset of 90° along the axial direction of said guide wire inner tube, resulting in said emission holes that are oriented at a 90° angle to each other.

7. The four-interface shock wave balloon catheter, as recited in claim 5, wherein said fluid discharge tube is arranged along said guide wire inner tube with an offset relative to said emission holes of said shock wave emitter.

8. The four-interface shock wave balloon catheter, as recited in claim 7, wherein the portion of said fluid discharge tube that extends into said balloon chamber is staggered relative to said emission holes when arranged around said guide wire inner tube.

9. The four-interface shock wave balloon catheter, as recited in claim 7, wherein the location where said shock wave emitters are arranged in said guide wire inner tube, is arranged with an insulating isolation layer that provides isolation between said conducting wires of said shock wave emitters and said distal segment.

10. The four-interface shock wave balloon catheter, as recited in claim 9, wherein said insulating isolation layer is arranged in said guide wire inner tube with said insulating isolation layer covering said guide wire inner tube.

11. The four-interface shock wave balloon catheter, as recited in claim 9, wherein said guide wire inner tube further comprises an insulating medium that covers said conducting wires, based on said insulating layer, said insulating isolation layer and said insulating medium, said portions of said conducting wires of said shock wave emitters, except for the portion that exposed to said emission holes, are insulating isolated from said balloon chamber and said fluid replenishment channel.

12. The four-interface shock wave balloon catheter, as recited in claim 11, wherein said insulating medium is arranged in said guide wire inner tube with said insulating medium covering said guide wire inner tube.

13. The four-interface shock wave balloon catheter, as recited in claim 12, wherein said fluid discharge tube is arranged within said guide wire inner tube with said insulating medium covering said guide wire inner tube in a segmented manner.

14. The four-interface shock wave balloon catheter, as recited in claim 12, wherein said shock wave balloon catheter further comprises at least one contrast marker which is directly or indirectly fixed to said distal segment within said balloon chamber, to monitor the position of said balloon during the procedure and ensure said balloon is located in the target calcified area of the blood vessel, based on the visualization of said contrast marker.

15. The four-interface shock wave balloon catheter, as recited in claim 14, the number of said contrast markers is two, said two contrast markers are directly or indirectly fixed to said distal segment at positions near the two opposite ends of said balloon within said balloon chamber, respectively.

16. The four-interface shock wave balloon catheter, as recited in claim 15, wherein said two contrast markers are arranged in a ring-shaped manner and are annularly disposed around said distal segment of said guide wire inner tube.

17. The four-interface shock wave balloon catheter, as recited in claim 16, wherein said contrast marker located near the end where said balloon is sealedly connected to said distal segment is fixed to said guide wire inner tube, while the other of said contrast marker, located near the end where said balloon is sealedly connected to said outer tube, is annularly disposed around said guide wire inner tube while being adhesively fixed to said insulating medium.

18. The four-interface shock wave balloon catheter, as recited in claim 16, wherein said guide wire interface of said catheter base is arranged coaxially with said catheter connection end, while said fluid replenishment interface, said fluid discharge interface, and said pulse interface are arranged laterally relative to said guide wire interface and said catheter connection end that are arranged coaxially, and are arranged such that said pulse interface is closer to said guide wire interface relative to said fluid replenishment interface and said fluid discharge interface.

19. A control system of a four-interface shock wave balloon catheter, comprising:
a catheter base, said catheter base is designed with a four-interface structure and has a fluid replenishment interface, a fluid discharge interface, a guide wire interface, a pulse interface, and a catheter connection end;
a shock wave balloon catheter, said shock wave balloon catheter comprises a guide wire inner tube, an outer tube, a balloon, a fluid discharge tube, and at least two shock wave emitters, wherein when said shock wave balloon catheter is connected to said catheter connection end of said catheter base, said outer tube is connected to said catheter connection end, said guide wire inner tube communicates with said guide wire interface through said catheter connection end, and extends out of said outer tube through said catheter connection end, forming a fluid replenishment channel between said guide wire inner tube and said outer tube, which communicates with said fluid replenishment interface through said catheter connection end, wherein the part of said guide wire inner tube that extends out of said outer tube is the distal segment of said guide wire inner tube, one end of said balloon is sealedly connected to the end of said outer tube that is farther from said catheter connection end, and the other end of said balloon is sealedly connected to said distal segment of said guide wire inner tube, forming a balloon chamber that communicates with said fluid replenishment channel, wherein said fluid discharge tube communicates with said fluid discharge interface through said catheter connection end, and into said balloon chamber along said guide wire inner tube, wherein said at least two shock wave emitters are arranged on said distal segment of said guide wire inner tube in said balloon chamber, said conducting wires of said at least two shock wave emitters, while being insulating isolated from said balloon chamber and said fluid replenishment channel, are arranged along said guide wire inner tube, and are led out through said catheter connection end at said pulse interface; and
a circulation control device, wherein said circulation control device comprises a fluid replenishment mechanism and a recovery mechanism, said fluid replenishment mechanism comprises a constant pressure peristaltic pump and a pressurizing pump, wherein said constant pressure peristaltic pump has a fluid replenishment port and an output port, and said output ports of said pressurizing pump and said constant pressure peristaltic pump are connected to said fluid replenishment interface of said catheter base through a three-way connector, wherein the pipeline of said pressurizing pump that connects said three-way connector is arranged with a three-way valve configured as an electromagnetic valve, where two of the interfaces of said three-way valve are respectively connected to said pressurizing pump and said three-way connector, and the third interface of said three-way valve serves as an air inlet, on the connecting pipe between said three-way connector and said output port of said constant pressure peristaltic pump, a first electromagnetic valve is installed to control the opening and closing of this connecting pipeline, wherein a pressure sensor is installed on the connecting pipeline between said three-way connector with said first electromagnetic valve and said output port of said constant pressure peristaltic pump, the recovery mechanism comprises a one-way valve and a second electromagnetic valve, wherein the inlet of said one-way valve is connected to said fluid discharge interface of said catheter base, the outlet of said one-way valve is connected to one of the interfaces of said second electromagnetic valve, and the other interface of said second electromagnetic valve serves as a fluid discharge port.

20. The control system of a four-interface shock wave balloon catheter, as recited in claim 19, wherein said fluid discharge port is directly or indirectly connected to said fluid replenishment port of said constant pressure peristaltic pump through a filtering device, to achieve recycling and reuse by filtering the waste fluid.
